(19) United States
(12) Patent Application Publication
(10) Pub. No.: US 2014/0079658 A1
(43) Pub. Date: Mar. 20, 2014

(54) AQUEOUS HAIR CLEANSING AGENT

(71) Applicant: KAO CORPORATION, Tokyo (JP)

(72) Inventors: Hiroyuki TERAZAKI, Sumida-ku (JP); Azusa KASUGA, Yoshikawa-shi (JP)

(73) Assignee: KAO CORPORATION, Tokyo (JP)

(21) Appl. No.: 13/727,342

(22) Filed: Dec. 26, 2012

(30) Foreign Application Priority Data
Sep. 20, 2012 (JP) 2012-207649

(51) Int. Cl. A61K 8/46; A61K 8/33; A61Q 5/02; A61K 8/86; A61K 8/365

(52) U.S. Cl. CPC A61K 8/466; A61K 8/86; A61K 8/365; A61Q 5/02; A61K 8/33
USPC 424/70.11; 424/70.24; 424/70.19

(57) ABSTRACT

The invention relates to an aqueous hair cleansing agent comprising the following components (A), (B), (C), and water and having a pH of 2 to 5 at 25° C. when diluted 20-fold by mass with water:

(A) an internal olefin sulfonate,
(B) an organic solvent selected from the group consisting of the following (B1) to (B5):
(B1) an aromatic alcohol,
(B2) a polypropylene glycol,
(B3) a N-alkyl pyrrolidone,
(B4) an alkylene carbonate,
(B5) a lactone or a cyclic ketone, and
   (C) an organic carboxylic acid selected from the group consisting of a hydroxymonocarboxylic acid and dicarboxylic acid.

### FIELD OF THE INVENTION

The present invention relates to an aqueous hair cleansing agent.

### BACKGROUND OF THE INVENTION

Hair tends to be put in the state of being dried out because it is always affected by ultraviolet rays and heat from sunlight and dryness and the like, and also, for example, daily shampooing, brushing, and heat from a drier promote such a tendency. In recent years where hair damages have been normalized due to the spread of coloring agents and perm agents, hair is damaged and friction of hair surface is increased. This is the reason why hairs are easily tangled with each other, which tends to bring about an unpleasant feeling to the touch during washing of the hair and combing difficulty after drying the hair. In light of this, improving rinsing characteristics, hair qualities such as feeling to the touch after rinsing and drying becomes important for the hair cleansing agent in addition to fundamental functions necessary for the hair cleansing agent such as foamability and detergency.

Patent Document 1 discloses a hair cleansing agent including an anionic surfactant containing a sulfuric acid residue, hydroxymonocarboxylic acid, and a specific organic solvent, the hair cleansing agent having a buffer capacity and also being superior in foamability besides shininess, softness, and tidy/manageable properties of hair. Patent Document 2 discloses a composition which contains a specific internal olefin sulfonic acid and is improved in foamability and feeling to the touch without any creak feeling when used as a shampoo.

### CITATION LIST

### Patent Document

Patent Document 1: JP-A-2003-252731
Patent Document 2: JP-A-2003-81935

### SUMMARY OF THE INVENTION

The present invention provides an aqueous hair cleansing agent comprising the following components (A), (B), (C), and water and having a pH of 2 to 5 at 25° C. when diluted 20-fold by mass with water:

(A) an internal olefin sulfonate;
(B) one or two or more organic solvents selected from the group consisting of the following (B1) to (B5);

(B1) an aromatic alcohol represented by the following formula (1):

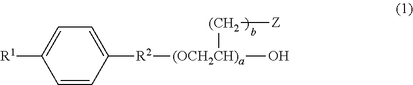

wherein R¹ represents a hydrogen atom, a methyl group, or a methoxy group, R² represents a single bond or a straight-chain or a branched chain alkylene group or alkenylene group having one to three carbon atoms, Z represents a hydrogen atom or a hydroxyl group, and a and b respectively denote a number from 0 to 5;

(B2) a N-alkyl pyrrolidone in which an alkyl group having 1 to 18 carbon atoms is bound with a nitrogen atom;

(B3) an alkylene carbonate having 1 to 4 carbon atoms;

(B4) a polypropylene glycol having a molecular weight of 200 to 1000; and

(B5) a lactone or a cyclic ketone represented by the following formula (2), (3), or (4):

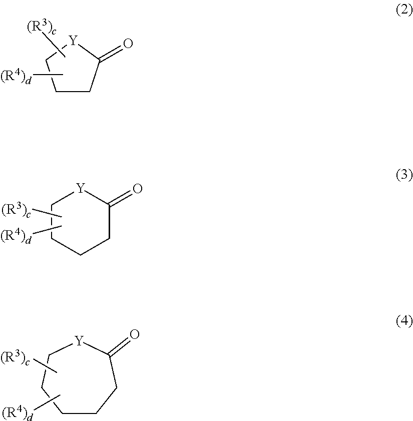

wherein Y represents a methylene group or an oxygen atom, R³ and R⁴ represent substituents differing from each other, and c and d denote a number from 0 or 1; and
(C) one or two or more organic carboxylic acids selected from the group consisting of a hydroxymonocarboxylic acid and dicarboxylic acid.

### DETAILED DESCRIPTION OF THE INVENTION

Conventional technologies typified by the aforementioned Patent Documents all fail to fulfill the performance required in each stage of foaming, rinsing, and after drying, that is, the capabilities of giving good foamability and good rinsing characteristics to detergents and imparting shininess to hair after drying.

Accordingly, the present invention relates to an aqueous hair cleansing agent having excellent performance in all of sufficient formability during washing, rinsing characteristics in rinsing, and shininess of hair after drying.

The aqueous hair cleansing agent of the present invention includes component (A): a sulfonate of an internal olefin (hereinafter referred to as “internal olefin sulfonate”) in terms of improving foaming (foamability), and imparting good rinsing characteristics and shininess to hair.

In the present invention, the internal olefin sulfonate which is the component (A) comprises:

(A1) an alkene sulfonate containing a sulfonyl group on any of the carbon atoms of a straight-chain alkene having 12 or more and 24 or less carbon atoms excluding the terminal carbon atoms or its salt;
(A2) a hydroxyalkanesulfonic acid containing a sulfonyl group on any one of the carbon atoms of a straight-chain hydroxyalkane having 12 or more and 24 or less carbon atoms excluding the terminal carbon atoms or its salt; or

a mixture of them.

In the present invention, the internal olefin means an olefin having a double bond inside (namely, except for terminal carbon atoms) of the carbon chain. The internal olefin sulfonate which is the component (A) is obtained, for example, by sulfonating a straight-chain alkene having 12 or more and 24 or less carbon atoms which contains a double bond on a carbon atom except for the terminal carbon atoms, followed by neutralization and hydrolysis. Specifically, when sulfonating an internal olefin, β-sartone is quantitatively produced and a part of β-sartone is converted into γ-sartone and alkenesulfonic acid, which are further converted into a hydroxyalkane sulfonate and alkene sulfonate through neutralizing/hydrolyzing processes (see, for example, J. Am. Oil Chem. Soc., 69, 39 (1992)). Here, the hydroxy group of the obtained hydroxyalkane sulfonate exists inside (except for terminal carbon atoms) of the alkane chain and the double bond of the olefin sulfonate exists inside (except for terminal carbon atoms) of the olefin chain. Also, the obtained product is primarily a mixture of these compounds. The “internal olefin sulfonate” which is the component (A) in this specification is a generic name of each of these products and a mixture of these products.

The number of carbon atoms of the internal olefin sulfonate which is the component (A) is 12 or more, preferably 14 or more, and more preferably 16 or more from the viewpoint of foamability and rinsing characteristics, and also from the viewpoint of imparting shine to hair. Also, the number of carbon atoms is 24 or less, preferably 20 or less, and more preferably 18 or less from the viewpoint of softness in rinsing. Also, the number of carbon atoms is 12 or more and 20 or less and preferably 12 or more and 18 or less from the viewpoint of foamability and rinsing characteristics, and also from the viewpoint of imparting shine to hair.

Among the above internal olefin sulfonates, those having 16 carbon atoms and those having 18 carbon atoms are preferable from the viewpoint of foamability, and these olefin sulfonates are preferably mixed prior to use. In this case, the ratio by mass of the content of the internal olefin sulfonate having 16 carbon atoms to the content of the internal olefin sulfonate having 18 carbon atoms (internal olefin sulfonate having 16 carbon atoms/internal olefin sulfonate having 18 carbons) is preferably 50/50 to 99/1, more preferably 60/40 to 95/5, even more preferably 70/30 to 90/10, even more preferably 75/25 to 90/10, and even more preferably 75/25 to 85/15 from the viewpoint of improving detergency, foam texture, foamability, rinsing characteristics, and feeling to the touch after rinsing.

In this case, the above ratio by mass can be measured by high-performance liquid chromatography mass spectrometer (hereinafter abbreviated as “HPLC-MS”). Specifically, the internal olefin sulfonates having 16 and 18 carbon atoms are separated from each other by HPLC and each internal olefin sulfonate is subjected to MS, whereby each internal olefin sulfonate can be identified and also, the ratio by mass can be found from the HPLC-MS peak areas.

The total ratio of the internal olefin sulfonates having 16 carbon atoms and 18 carbon atoms in the total amount of the component (A) is preferably 50% by mass or more, more preferably 60% by mass or more, even more preferably 70% by mass or more, even more preferably 80% by mass or more, even more preferably 90% by mass or more, and even more preferably 95% by mass or more from the viewpoint of improving foamability and rinsing characteristics.

In the present invention, the sulfonic acid group of the internal olefin sulfonate contained in the component (A) is located inside of the olefin chain or alkane chain. In this case, it is desirable from the point of foamability that the content of the internal olefin sulfonate with the sulfonic acid group located at the second position of the olefin chain or alkane chain be low whereas the content of the internal olefin sulfonate with the sulfonic acid group located at the third or more inward position be high. In the case where the internal olefin sulfonate having 16 carbon atoms and the internal olefin sulfonate having 18 carbon atoms coexist in the component (A), both of the internal olefin sulfonate having 16 carbon atoms and the internal olefin sulfonate having 18 carbon atoms are preferably those in which the content of the internal olefin sulfonate with the sulfonic acid group located at the second position of the olefin chain or alkane chain is low.

The ratio of a material with the sulfonic acid group being located at the second position in the component (A) is preferably 20% by mass or less, more preferably less than 20% by mass, even more preferably 19% by mass or less, even more preferably less than 18% by mass, and even more preferably 17.5% by mass or less from the viewpoint of foamability and rinsing characteristics. Also, the lower limit of the above ratio is preferably 5% by mass or more, more preferably 6% by mass or more, even more preferably 7% by mass or more, even more preferably 8% by mass or more, and even more preferably 9% by mass or more from the viewpoint of reduction in production cost and improvement in productivity. Here, the position where the sulfonic acid group is located depends on the position of a double bond in the internal olefin used as starting material and the ratio of the presence of the sulfonic acid group located at the second position almost accords to the ratio of the presence of a double bond present at the second position of the raw material internal olefin. In this case, the content of the internal olefin sulfonate with the sulfonic acid group being located at the second position in the internal olefin sulfonate can be measured by a method such as nuclear magnetic resonance spectrum. Furthermore, the content of the internal olefin sulfonate with the sulfonic acid group being located at the third or more inward position of the olefin chain or alkane chain in the component (A) is preferably 70% by mass or more, more preferably 75% by mass or more, and even more preferably 80% by mass or more from the viewpoint of improvements in foamability, foam texture, and rinsing characteristics. Moreover, the content of the internal olefin sulfonate with the sulfonic acid group being located at the first position of the olefin chain or alkane chain is preferably 3% by mass or less.

Also, in the component (A), (A1) the alkenesulfonic acid or its salt and (A2) the hydroxyalkanesulfonic acid or its salt may be used as a mixture of them depending on the performance to be required. When the mixture is used, the ratio by mass (A2)/(A1) of the component (A2) to the component (A1) is preferably 50/50 to 95/5 and more preferably 50/50 to 90/10.

Examples of the salt when the component (A) is the internal olefin sulfonate salt include alkali metal salts such as a sodium salt and potassium salt; alkali earth metal salts such as a calcium salt and magnesium salt; ammonium salts; and organic ammonium salts generated from a monoethanolamine, diethanolamine, triethanolamine, or the like. Among these compounds, alkali metal salts and ammonium salts are preferable from the point of temperature stability, easy familiarity, and coloring resistance with time.

The content of the component (A) in the aqueous hair cleansing agent of the present invention is preferably 1% by mass or more, more preferably 2% by mass or more, and even more preferably 5% by mass or more, and preferably 20% by mass or less, more preferably 18% by mass or less, and even more preferably 15% by mass or less from the viewpoint of rinsing characteristics and shininess. Also, the content of the component (A) in the aqueous hair cleansing agent of the present invention is preferably 1% by mass or more and 20% by mass or less, more preferably 2% by mass or more and 18% by mass or less, and even more preferably 5% by mass or more and 15% by mass or less from the viewpoint of smoothness in rinsing and from the viewpoint of imparting shininess to hair.

The internal olefin sulfonate which is the component (A) may be produced by sulfonating an internal olefin having 12 to 24 carbon atoms, followed by neutralization and hydrolysis. No particular limitation is imposed on the conditions of sulfonation, neutralization, and hydrolysis, and these conditions may be determined on referring to the method described in Japanese Patent No. 1633184, Japanese Patent No. 2625150, Tenside Surf. Det., 31(5), 299 (1994).

As mentioned above, the internal olefin in the present invention means an olefin containing a double bond inside (except for the terminal carbon atoms) of the carbon chain. However, the internal olefin used as the raw material may be one partly containing, as impurities, an α-olefin and paraffin component having a double bond at its carbon chain terminal. The number of carbon atoms of the internal olefin is 12 or more and 24 or less, preferably 12 or more and 20 or less, even more preferably 12 or more and 18 or less from the viewpoint of foamability and rinsing characteristics. The internal olefins may be used either singly or in combinations of two or more.

The total content of the internal olefins containing a double bond at the second position among the internal olefins used as the raw material is preferably 20% by mass or less, more preferably less than 20% by mass, even more preferably 19% by mass or less, even more preferably 18% by mass or less, and even more preferably 17.5% by mass or less from the viewpoint of foamability and rinsing characteristics. Also, the above total content is preferably 5% by mass or more, more preferably 6% by mass or more, even more preferably 7% by mass or more, even more preferably 8% by mass or more, and even more preferably 9% by mass or more from the viewpoint of reduction in production cost and improvement in productivity.

The content of an α-olefin with a double bond being located at the first position in the raw material internal olefin is preferably 3.0% by mass or less, more preferably less than 2.8% by mass, even more preferably 2.5% by mass or less, and even more preferably 2.0% by mass or less and it is undesirable to contain such an α-olefin from the viewpoint of foamability and smoothness in rinsing and also from the viewpoint of imparting shine to hair. It is however preferable that the content be 0.1% by mass or more from the viewpoint of reducing production cost and improving productivity.

Moreover, the total content of the internal olefins with double bonds being located on the third or more inward positions in the raw material internal olefin is preferably 70% by mass or more, more preferably 75% by mass or more, and even more preferably 80% by mass or more from the viewpoint of foamability and smoothness in rinsing and also from the viewpoint of imparting shine to hair.

The ratio of the positions of double bonds in the raw material internal olefin may be measured by, for example, a gas chromatographic mass spectrometer (hereinafter abbreviated as GC-MS). Specifically, components differing from each other in carbon chain length and the position of a double bond are exactly separated from each other by a gas chromatographic analyzer (hereinafter abbreviated as GC) and each subjected to a mass spectrometer (hereinafter abbreviated as MS), thereby enabling identification of the position of each double bond and the ratio of each double bond from the GC peak area.

The raw material internal olefin may contain a paraffin component. The content of the paraffin component is preferably 10% by mass or less, more preferably 5% by mass or less, and even more preferably 3% by mass or less, and it is undesirable to contain such a paraffin component from the viewpoint of foamability. It is however preferable that the content of the paraffin component be 0.001% by mass or more from the viewpoint of reducing cost and improving productivity. The content of the paraffin component can be measured by, for example, GC-MS and specifically found by the method described in the examples.

The component (B) is one or two or more organic solvents selected from the group consisting of the following (B1) to (B5).

(B1) An aromatic alcohol represented by the following formula (1):

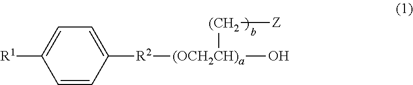

wherein R¹ represents a hydrogen atom, a methyl group, or a methoxy group, R² represents a single bond or a straight-chain or branched alkylene group or alkenylene group having 1 to 3 carbon atoms, Z represents a hydrogen atom or a hydroxyl group, and a and b denote a number from 0 to 5;
(B2) a N-alkyl pyrrolidone in which an alkyl group having 1 to 18 carbon atoms is bound with a nitrogen atom;
(B3) an alkylene carbonate having 1 to 4 carbon atoms;
(B4) a polypropylene glycol having a molecular weight of 200 to 1000; and
(B5) a lactone or cyclic ketone represented by the formula (2), (3), or (4):

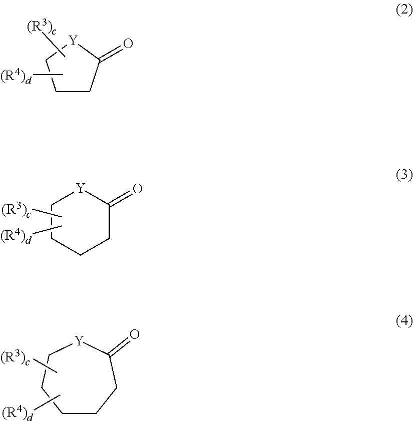

wherein Y represents a methylene group or an oxygen atom, R³ and R⁴ represent substituents different from each other, and c and d denote a number of 0 or 1.

These components (B) may be used either singly or in combinations of two or more.

Examples of the aromatic alcohol which is the component (B1) among these components (B) include benzyl alcohol, cinnamyl alcohol, phenethyl alcohol, p-anisyl alcohol, p-methylbenzyl alcohol, phenoxyethanol, and 2-benzyloxyethanol. Among these alcohols, benzyl alcohol and 2-benzyloxyethanol are preferable. Examples of the component (B2) include N-methylpyrrolidone, N-octyl pyrrolidone, and N-lauryl pyrrolidone. Examples of the component (B3) include ethylene carbonate and propylene carbonate. As (B4) a polypropylene glycol, those having a molecular weight of 300 to 400 are preferable. As R³ and R⁴ in the formulae (2) to (4) in the component (B5), a straight-chain, branched, or cyclic alkyl group, hydroxyl group, sulfonic acid group, phosphoric acid group, carboxyl group, phenyl group, sulfoalkyl group, alkyl phosphate group, and carboxyalkyl group are preferable. Particularly, a straight-chain or branched alkyl group having 1 to 6 carbon atoms, for example, a methyl group, ethyl group, propyl group, isopropyl group, or butyl group which is substituted at the γ-position in the case of γ-lactone and at the δ-position (namely, methylene adjacent to a hetero oxygen atom) in the case of δ-lactone is preferable. Also, in the case of intending to increase the water-solubility of the compounds (2) to (4), it is preferable to include an acidic group such as a sulfonic acid group, phosphoric acid group, or carboxy group or an alkyl group substituted with these acid groups as R³ or R⁴. Examples of the lactone among the component (B5) include γ-butyrolactone, γ-caprolactone, γ-valerolactone, δ-valerolactone, δ-caprolactone, and δ-heptanolactone. It is preferable to use γ-lactone, γ-butyrolactone, or γ-caprolactone among these lactone compounds in view of stability. Examples of the cyclic ketone among the component (B5) include cyclopentanone, cyclohexanone, cycloheptanone, and 4-methyl cycloheptanone.

Also, as the component (B), one or more types selected from benzyl alcohol, 2-benzyloxyethanol, and polypropylene glycol having a molecular weight of 200 to 700 are preferably used.

The content of the component (B) as the total amount of the components (B1) to (B5) is preferably 0.01% by mass or more, more preferably 0.05% by mass or more, and even more preferably 0.1% by mass or more based on the total amount of the aqueous hair cleansing agent from the viewpoint of smoothness in rinsing and also from the viewpoint of imparting shine to hair. The content of the component (B) as the total amount of the components (B1) to (B5) is preferably 20% by mass or less, more preferably 10% by mass or less, and even more preferably 5% by mass or less.

The component (C) is one or two or more organic carboxylic acids selected from hydroxymonocarboxylic acids and dicarboxylic acids. Specific examples of the hydroxymonocarboxylic acids include glycolic acid, lactic acid, glyceric acid, gluconic acid, and pantothenic acid. Specific examples of the dicarboxylic acid include malic acid, tartaric acid, oxalic acid, malonic acid, maleic acid, succinic acid, and glutaric acid. It is preferable to use one or more types selected from the group consisting of glycolic acid, lactic acid, malic acid, and tartaric acid from the viewpoint of foamability.

The content of these components (C) is preferably 0.3% by mass or more, more preferably 0.5% by mass or more, and even more preferably 0.7% by mass or more relative to the total amount of the aqueous hair cleansing agent of the present invention from the viewpoint of improvement in shininess after drying. Also, the content of the component (C) is preferably 5% by mass or less, more preferably 3% by mass or less, and even more preferably 2% by mass or less relative to the total amount of the aqueous hair cleansing agent of the present invention from the viewpoint of foamability.

The aqueous hair cleansing agent of the present invention has a pH of 2 to 5 at 25° C. when diluted 20-fold by mass with water. The use of a combination of the components (A) to (C) and the above pH enables an improvement in shininess after drying. From the above point of view, the pH of a solution of the hair cleansing agent at 25° C. when the aqueous hair cleansing agent of the present invention is diluted 20-fold by mass with water is preferably 2.5 to 4.5 and more preferably 3 to 4.5.

The content of the components (A) to (C) in the aqueous hair cleansing agent of the present invention is preferably as follows: component (A): 1 to 20% by mass, component (B): 0.05 to 10% by mass, and component (C): 0.5 to 3% by mass.

Also, the content of water in the aqueous hair cleansing agent of the present invention is preferably 50% by mass or more and 95% by mass or less, more preferably 60% by mass or more and 90% by mass or less.

The aqueous hair cleansing agent of the present invention may include components shown below besides the above components (A) to (C) and water.

The aqueous hair cleansing agent of the present invention preferably contains a polyoxyethylene alkyl ether sulfate represented by the following formula (5) as an anionic surfactant other than the component (A) and also as a component (D).

R⁵O(CH₂CH₂O)ₑSO₃M (5)

R⁵ represents an alkyl group or an alkenyl group having 10 to 18 carbon atoms, M represents an alkali metal, an alkali earth metal, ammonium, an alkanol amine, or a basic amino acid, and e represents an average addition molar number of ethylene oxide and denotes a number from 0.5 to 5 in an average by mass.

Among these compounds represented by the above formula, those in which R⁵ in the formula (5) is an alkyl group having 12 to 14 carbon atoms are preferable for the viewpoint of achieving a balance between quick foamability and good touch of foam. Also, the average addition molar number e is preferably 0.9 to 4 and more preferably 1 to 3. The polyoxyethylene alkyl ether sulfate obtained when M is ammonium or sodium in the above formula is preferable.

The content of the component (D) is so designed that the total amount of the components (A) and (D) is preferably 3% by mass or more, more preferably 5% by mass or more, and even more preferably 7% by mass or more relative to the total amount of the aqueous hair cleansing agent from the viewpoint of more improving foamability. Also, the total amount of the components (A) and (D) is preferably 20% by mass or less, more preferably 19%, by mass or less, and even more preferably 18% by mass or less relative to the total amount of the aqueous hair cleansing agent from the viewpoint of improving rinse-off characteristics and suppressing a residual feeling in rinsing.

The aqueous hair cleansing agent of the present invention may include a nonionic surfactant selected from a glyceryl ether represented by the following formula (6) and a polyoxyalkylene alkyl ether represented by the following formula (7) as a component (E) to more improve foamability.

R⁶—O—CH₂—CH(OH)—CH₂—OH (6)

R⁶ represents a straight-chain or branched hydrocarbon group having 1 to 22 carbon atoms.

As the glyceryl ether, R⁶ in the formula (6) is a straight-chain or branched hydrocarbon group having, preferably, 2 to 22, more preferably 4 to 20, and more preferably 6 to 16 carbon atoms. Specific examples of a desired glyceryl ether compound include 2-ethylhexyl glyceryl ether and isodecyl glyceryl ether.

R⁷O—(PO)ₙ/(EO)ₘ—R⁸ (7)

R⁷ represents a straight-chain alkyl group or alkenyl group having 8 to 10 carbon atoms, PO represents a propyleneoxy group, EO represents an ethyleneoxy group, n which is an average addition molar number denotes a number from 1.5 to 3.0, m which is an average addition molar number denotes a number from 0 to 1.0, and R⁸ represents a hydrogen atom or a methyl group.

Examples of the polyoxyalkylene alkyl ether include a polyoxypropylene (3) monooctyl ether.

Glyceryl ethers and polyoxyalkylene alkyl ethers may be used either singly or in combinations of two or more. When the aqueous hair cleansing agent of the present invention is used in an aqueous liquid detergent form, it is not only more improved in foamability but also moderate liquid characteristic is obtained if a monoalkyl glyceryl ether is used together with the component (A).

The components (E) may be used either singly or in combinations of two or more. The content of the component (E) is preferably 0.01 to 20% by mass, more preferably 0.05 to 15% by mass, and more preferably 0.1 to 10% by mass in the total amount of the composition from the viewpoint of quickness of foaming.

The aqueous hair cleansing agent of the present invention may further contain an amphoteric surfactant as a component (F) to improve foamability.

Examples of the amphoteric surfactant include betaine type surfactants. Among these surfactants, an alkyldimethylaminoacetic acid betaine, fatty acid amidopropyl betaine, and alkylhydroxy sulfobetaine are preferable and particularly, fatty acid amidopropyl betaine is preferable. As the fatty acid amidopropyl betaine, those having an acyl group having 8 to 18 and preferably 10 to 16 carbon atoms are preferable and particularly, lauric acid amidopropyl betaine, palm kernel oil fatty acid amidopropyl betaine, coconut oil fatty acid amidopropyl betaine, and the like are preferable.

Examples of other amphoteric surfactant include sultaine type surfactants such as laurylhydroxy sultaine.

These amphoteric surfactants may be used in the aqueous hair cleansing agent either singly or in combinations of two or more. When the aqueous hair cleansing agent of the present invention is used in an aqueous liquid detergent form, it is not only more improved in foamability but also moderate liquidness is obtained if a fatty acid amidopropyl betaine is used together with the component (A).

The content of the amphoteric surfactant is preferably 0.01 to 15% by mass, more preferably 0.05 to 8% by mass, and even more preferably 0.1 to 6% by mass relative to the total amount of the aqueous hair cleansing agent of the present invention from the viewpoint of producing a good foam-increasing effect.

The aqueous hair cleansing agent of the present invention may comprise a cationized polymer. Examples of the cationized polymer include cationized cellulose derivatives such as cationized hydroxyethyl cellulose; cationic starch; cationized galactomannan such as cationized fenugreek gum, cationized guar gum, cationized tara gum, and cationized locust bean gum, and their derivatives; copolymers such as a diallyl quaternary ammonium salt/acrylamide copolymer, vinylimidazolium trichloride/vinyl pyrrolidone copolymer, hydroxyethyl cellulose/dimethyldiallylammonium chloride copolymer, vinyl pyrrolidone/quaternized dimethylaminoethylmethacrylate copolymer, polyvinyl pyrrolidone/alkylaminoacrylate copolymer, polyvinyl pyrrolidone/alkylaminoacrylate/vinyl caprolactam copolymer, vinyl pyrrolidone/methacrylamidopropyltrimethylammonium chloride copolymer, alkylacrylamide/acrylate/alkylaminoalkylacrylamide/polyethylene glycol methacrylate copolymer, and adipic acid/dimethylaminohydroxypropylethylenetriamine copolymer (manufactured by Sandoz, Cartaretin), cationic polymers described in Japanese Patent Application Laid-Open No. 53-139734 and cationic polymers described in Japanese Patent Application Laid-Open No. 60-36407.

Among these compounds, one or more types selected from the group consisting of cationized guar gum, cationized tara gum, and cationized hydroxyethyl cellulose are preferably used from the viewpoint of reducing stickiness and a squeaky feeling in rinsing.

Among these compounds, the above cationized galactomannan is a water-soluble cationized polymer obtained by introducing a quaternary nitrogen-containing group into a galactomannan consisting of a principal chain including mannose as a structural unit and a side chain constituted of a galactose unit. This galactomannan is obtained from, for example, albumen of a seed of a leguminous plant. A galactomannan in which the ratio of galactose to mannose is 1:1 is fenugreek gum, a galactomannan in which the ratio of galactose to mannose is 1:2 is guar gum, a galactomannan in which the ratio of galactose to mannose is 1:3 is tara gum, and a galactomannan in which the ratio of galactose to mannose is 1:4 is locust bean gum.

Examples of commercially available products of the cationized galactomannan include Catinal CF-100 (trade name, manufactured by Toho Chemical Industries, Co., Ltd.) as the cationized fenugreek gum. Examples commercially available products of the cationized guar gum include Jagaur Series such as Jagaur C-13S, Jagaur-C-14S, and Jagaur C-17 (trade name, manufactured by Rhodia Japan, guar hydroxypropyltriammonium chloride). Also, examples of commercially available products of the cationized tara gum include Catinal CTR-100 and Catinal CTR-200 (trade name, manufactured by Toho Chemical Industries, Co., Ltd.). Also, examples of commercially available products of the cationized locust bean gum include Catinal CLB-100 (trade name, manufactured by Toho Chemical Industries, Co., Ltd., locust bean hydroxypropyl trimonium chloride).

Examples of other commercially available products of the cationized polymer include Merquat 550 (trade name, manufactured by Nalco Chemical Co., Ltd., copolymer of acrylamide and diallyldimethylammonium salt; INCI name: POLYQUATERNIUM-7), Luviquat FC370 (trade name, manufactured by BASF Corp., copolymer of 1-vinyl-2-pyrrolidone and 1-vinyl-3-methylimidazolium salt; INCI name: POLYQUATERNIUM-16), Gafquat 757N (trade name, manufactured by ISP Corp., copolymer of 1-vinyl-2-pyrrolidone and dimethylaminoethylmethacrylate; INCI name: POLYQUATERNIUM-11), Ucare Polymer JR series and Ucare Polymer LR series (trade name, manufactured by Amerchol Corp., salt of a reaction product between trimethylammonium-substituted epoxide and hydroxyethyl cellulose; INCI name: POLYQUATERNIUM-10), and POIZ C-60H, POIZ C-80M, and POIZ C-150L (trade name, manufactured by Kao Corporation, salt of a reaction product between trimethylammonium-substituted epoxide and hydroxyethyl cellulose; INCI name: POLYQUATERNIUM-10).

These cationized polymers may be used either singly or in combinations of two or more and the content of these cationized polymers may be 0.01 to 3% by mass, preferably 0.02 to 2% by mass, and more preferably 0.05 to 1% by mass relative to the total amount of the aqueous hair cleansing agent of the present invention from the viewpoint of reducing a squeaky feeling in rinsing.

A cationic surfactant or silicones may further be formulated in the aqueous hair cleansing agent of the present invention to obtain better finish after drying.

Examples of the cationic surfactant include (i) an alkyltrimethylammonium salt, (ii) an alkoxytrimethylammonium salt, (iii) a dialkyldimethylammonium salt, (iv) an alkyldimethylamine and its salt, (v) an alkoxydimethylamine and its salt, and (vi) an alkylamidodimethylamine and its salt.

(i) Alkyltrimethylammonium Salt

Examples of the alkyltrimethylammonium salt include compounds represented by the following formula.

R¹¹—N⁺(CH₃)₃X⁻

R¹¹ represents an alkyl group having 12 to 22 carbon atoms, X⁻ represents a halide ion such as a chloride ion or a bromide ion.

More specific examples of the alkyl trimethylammonium salt include cetyltrimethylammonium chloride, stearyltrimethylammonium chloride, and behenyltrimethylammonium chloride.

(ii) Alkoxytrimethylammonium Salt

Examples of the alkoxytrimethylammonium salt include compounds represented by the following formula.

R¹²—O—R¹³—N⁺(CH₃)₃X⁻

R¹² represents an alkyl group having 12 to 22 carbon atoms, R¹³ represents an ethylene group or propylene group which may be substituted with a hydroxy group, and X⁻ is the same as above.

More specific examples of the alkoxytrimethylammonium salt include stearoxypropyltrimethylammonium chloride, stearoxyethyltrimethylammonium chloride, and stearoxyhydroxypropyltrimethylammonium chloride.

(iii) Dialkyldimethylammonium Salt

Examples of the dialkyldimethylammonium salt include compounds represented by the following formula.

(R¹⁴)₂N⁺(CH₃)₂Z⁺

R₁₄s respectively represent an alkyl group having 12 to 22 carbon atoms or a benzyl group and X⁻ is the same as above).

More specific examples of the dialkyl dimethylammonium salt include distearyldimethylammonium chloride.

(iv) Alkyldimethylamine and its Salt

Examples of the alkyldimethylamine and its salt include compounds represented by the following formula and their salts.

R¹⁵—N(CH₃)₂

R¹⁵ represents an alkyl group having 12 to 22 carbon atoms.

More specific examples of the alkyldimethylamine and its salt include behenyldimethylamine, stearyldimethylamine, and their organic acid salts.

(v) Alkoxydimethylamine and its Salt

Examples of the alkoxydimethylamine and its salt include compounds represented by the following formula and their salts.

R¹⁶—O—R¹⁷—N(CH₃)₂

R¹⁶ represents an alkyl group having 12 to 22 carbon atoms and R¹⁷ represents an ethylene group or a propylene group.

(vi) Alkylamidodimethylamine and its Salt

Examples of the alkylamidodimethylamine and its salt include compounds represented by the following formula and their salts.

R¹⁸—C(═O)NH—R¹⁹—N(CH₃)₂

R¹⁸ represents an alkyl group having 11 to 21 carbon atoms and R¹⁹ represents an ethylene group or a propylene group.

Examples of cationic surfactants other than the above cationic surfactants (i) to (vi) include lanolin fatty acid aminopropylethyldimethylammonium ethylsulfate (ethyl sulfate of alkanoylaminopropyldimethylethylammonium, in which the alkanoyl group is derived from lanolin), lanolin fatty acid aminoethyltriethylammonium ethylsulfate, lanolin fatty acid aminopropyltriethylammonium ethylsulfate, lanolin fatty acid aminoethyltrimethylammonium methylsulfate, lanolin fatty acid aminopropylethyldimethylammonium methylsulfate, isoalkanoic acid (C14-20) aminopropylethyldimethylammonium ethylsulfate, isoalkanoic acid (C18-22) aminopropylethyldimethylammonium ethylsulfate, isostearic acid aminopropylethyldimethylammonium ethylsulfate, isononanoic acid aminopropylethyldimethylammonium ethylsulfate, and alkyltrimethylammonium saccharin.

These cationic surfactants may be used in combinations of two or more and the content of these cationic surfactants is preferably 0.01 to 10% by mass, more preferably 0.02 to 6% by mass, and even more preferably 0.05 to 3% by mass in the aqueous hair cleansing agent of the present invention from the point of hair smooth texture since shampooing until rinsing.

Examples of the silicones include the following compounds.

### (I) Dimethylpolysiloxane

R²⁰(CH₃)₂SiO—[(CH₃)₂SiO]_{y}—Si(CH₃)₂R²⁰

R²⁰s respectively represent a methyl group or a hydroxy group, and y denotes a number from 1 to 20,000.

The average particle diameter of dispersed particles of dimethylpolysiloxane is preferably less than 100 μm, more preferably 50 μm or less, even more preferably 4 μm or less, and even more preferably 2 μm or less. Also, the average particle diameter is preferably 0.1 μm or more from the point of feeling on use and conditioning effect.

As this dimethylpolysiloxane, products commercially available under the name of, for example, “Silicone CF2450” (trade name, manufactured by Dow Corning Toray Co., Ltd.) which contains 60% by mass of a dimethylpolysiloxane oil represented by the above formula in which y is 300 to 6,500 and has an average particle diameter of 0.8 μm, “Silicone CF2460” (trade name, manufactured by Dow Corning Toray Co., Ltd.) which contains 50% by mass of a dimethylpolysiloxane represented by the above formula in which y is 300 to 6,500 and has an average particle diameter of 50 μm, or “KHE-1” (trade name, manufactured by Shin-Etsu Chemical Co., Ltd.) may be used.

### (II) Amino-Modified Silicone

A silicone compound which has an average molecular weight of about 3,000 to 100,000 and is described under the name of Amodimethicone in CTFA Dictionary (Cosmetic Ingredient Dictionary, USA), Third edition, is preferable as the amino-modified silicone, though various amino-modified silicones may be used. Examples of commercially available products include SM8704C (trade name, manufactured by Dow Corning Toray Co., Ltd.), DC929 (trade name, manufactured by Dow Corning Corporation), KT1989 (trade name, manufactured by Momentive Performance Materials Inc.), and 8500 Conditioning Agent, DOW CORNING TORAY SS-3588, DOW CORNING TORAY SILSTYLE 104 (trade name, manufactured by Dow Corning Toray Co., Ltd.).

### (III) Other Silicones

Other examples of the silicones include, besides the above compounds, a polyether-modified silicon, methylphenyl polysiloxane, fatty acid-modified silicone, alcohol-modified silicone, alkoxy-modified silicone, epoxy-modified silicone, fluorine-modified silicone, cyclic silicone, and alkyl-modified silicone.

These silicones may be used in combinations of two or more and the content of these silicones is 0.01 to 10% by mass, more preferably 0.05 to 5% by mass, and even more preferably 0.1 to 3% by mass in the aqueous hair cleansing agent of the present invention from the point of smooth texture since shampooing until rinsing.

The aqueous hair cleansing agent of the present invention may further comprise a pearling agent containing an ethylene glycol mono-fatty acid ester, ethylene glycol di-fatty acid ester, ethylene glycol monoalkyl ether, or ethylene glycol dialkyl ether.

Examples of the ethylene glycol mono-fatty acid ester include ethylene glycol monostearate and ethylene glycol monobehenate, and examples of the ethylene glycol di-fatty acid ester include ethylene glycol distearyl ester (written as “ethylene glycol distearyl” in an example (Table 3) which will be explained later) and ethylene glycol dibehenyl ester. Examples of the ethylene glycol monoalkyl ether include ethylene glycol monostearyl ether. Also, examples of the ethylene glycol dialkyl ether include ethylene glycol distearyl ether.

These compounds may be used in combinations of two or more and the content of these compounds is preferably 0.1 to 10% by mass, more preferably 0.2 to 5% by mass, and even more preferably 0.5 to 4% by mass in the aqueous hair cleansing agent from the point of improving the preserving stability of the aqueous hair cleansing agent and smooth texture during foaming and rinsing.

Also, the aqueous hair cleansing agent of the present invention may include an oily agent as other conditioning agent. Examples of the oily agent include hydrocarbons such as squalene, squalane, liquid paraffin, liquid isoparaffin, and cycloparaffin; glycerides such as castor oil, cacao-seed oil, mink oil, avocado oil, olive oil, sunflower oil, and camellia oil; waxes such as beeswax, spermaceti, lanolin, and carnauba wax; alcohols such as cetyl alcohol, oleyl alcohol, stearyl alcohol, isostearyl alcohol, 2-octyldodecanol, glycerin, myristyl alcohol, behenyl alcohol, and cetostearyl alcohol; esters such as isopropyl palmitate, isopropyl myristate, octyldodecyl myristate, hexyllaurate, cetyllactate, propylene glycol monostearate, oleyl oleate, hexadecyl 2-ethylhexanoate, isononyl isononanoate, and tridecyl isononanoate; higher fatty acids such as capric acid, lauric acid, myristic acid, palmitic acid, stearic acid, behenic acid, oleic acid, coconut oil fatty acid, isostearylacid, and isopalmitic acid; and others including isostearyl glyceryl ether and polyoxypropylene butyl ether. Among these compounds, higher alcohols are preferable and particularly, myristyl alcohol, cetyl alcohol, stearyl alcohol, sunflower oil, and camellia oil are preferable.

These oily agents may be used either singly or in combinations of two or more and the content of these oily agents is preferably 0.001 to 2% by mass, more preferably 0.005 to 1.5% by mass, and even more preferably 0.01 to 1% by mass in the aqueous hair cleansing agent of the present invention.

The aqueous hair cleansing agent of the present invention may include a viscosity regulating agent and examples of the viscosity regulating agent include hydroxyethyl cellulose, methyl cellulose, polyethylene glycol, ethylene glycol, propylene glycol, isoprene glycol, ethanol, claymineral, and salts (for example, sodium chloride, ammonium chloride, and sodium citrate). Among these compounds, ethanol, sodium chloride, and sodium citrate are preferable. These viscosity regulating agents may be used in combinations of two or more and the use amount of these agents is preferably 0.01 to 5% by mass, more preferably 0.05 to 4% by mass, and even more preferably 0.1 to 3% by mass in the aqueous hair cleansing agent of the present invention from the point of the quantity of foam and foam texture.

The aqueous hair cleansing agent of the present invention may be properly formulated with components used for usual aqueous hair cleansing agent, besides the above components corresponding to the purpose of use. Examples of these components include anionic surfactants other than the components (A) and (D) (sulfate type anionic surfactants such as an alkyl sulfate, alkenyl sulfate, alkyl sulfosuccinate, alkyl polyoxyalkylenesulfosuccinate, and polyoxyalkylene alkylphenyl ether sulfate which do not correspond to the component (D), sulfonic acid type anionic surfactants such as an alkyl sulfosuccinate, alkyl polyoxyalkylenesulfosuccinate, and alkane sulfonate which do not correspond to the component (A), carboxylic acid type anionic surfactants such as a higher fatty acid salt, and alkyl ether carboxylic acid or its salt), nonionic surfactants other than the component (E) (for example, polyoxyalkylene sorbitan fatty acid esters, polyoxyalkylene sorbitol fatty acid esters, polyoxyalkylene glycerin fatty acid esters, polyoxyalkylene fatty acid esters, polyoxyalkylene alkylphenyl ethers, polyoxyalkylene (hydrogenated) caster oils, sucrose fatty acids esters, polyglycerin alkyl ethers, polyglycerin fatty acid esters, fatty acid alkanolamide, and alkyl glycosides), antidandruff agent, vitamins, disinfectant, anti-inflammatory agent (glycyrrhizic acid, glycyrrhetic acid, and their derivatives), antiseptic, chelating agent, moisturizing agent (for example, sorbitol and pantenol), colorants (for example, dyes and pigments), extracts (polar solvent extracts from a eucalyptus, shells having the nacreous layer or proteins obtained from pearls, or its hydrolysate, proteins obtained from honey, royal jelly, silk, or its hydrolysate, protein-containing extracts obtained from seeds of leguminous plant, Asian ginseng extracts, rice germ extracts, fucus vesiculosus extracts, aloe extracts, lotus leaf extracts, pomegranate extracts, rosehip extracts, matricaria extracts, glycyrrhiza extract, alpinia leaf extract, chlorella extracts, and the like), pearling agents such as titanium oxide other than the above components, perfumes, ultraviolet absorbers, antioxidants, shea butter, rose water, orange oil, and eucalyptus oil.

The aqueous hair cleansing agent of the present invention is produced by blending the components (A) to (C) with these other components and by dissolving or dispersing the obtained mixture in water.

A pH regulating agent ether than the above component (C) may be formulated in the aqueous hair cleansing agent of the present invention. Examples of the pH regulating agent like this include organic acids other than component (C) such as aromatic carboxylic acids such as benzoic acid, monocarboxylic acids such as acetic acid, and tricarboxylic acids such as citric acid, and inorganic acids such as phosphoric acid. Also, as other pH regulating agents, a base such as sodium hydroxide, potassium hydroxide, or ammonium chloride may be used together with these organic acids.

An appropriate form of the aqueous hair cleansing agent of the present invention is preferably a liquid form using water, or water and lower alcohol as a solvent though it may be selected from a liquid form, gel form, and the like.

While the present invention has been described in its preferred form, it is not limited to the above embodiments, and modifications, improvements and the like made within the range where the object of the present invention can be achieved are encompassed in the present invention.

### EXAMPLES

The present invention will be explained in more detail by way of examples, which are not intended to be limiting of the present invention. In these examples and comparative examples, all designations of “%” indicate “% by mass”, unless otherwise noted. Also, methods for measuring various properties are as follows.

### (1) Measuring Method

(i) Method for Measuring the Position of a Double Bond in a Raw Material Internal Olefin

The position of a double bond in the internal olefin was measured by using gas chromatography (hereinafter abbreviated as GC). Specifically, dimethyl disulfide was made to react with the internal olefin to form a dithionated derivative and then each component was separated by GC. Thus, the position of a double bond in the internal olefin was found from each peak area.

In this case, instruments used for the measurement and the condition of analysis are as follows.

GC instrument (trade name: HP6890, manufactured by Hewlett Packard), column (trade name: Ultra-Alloy-1HT Capillary Column 30 m×250 μm×0.15 μm, manufactured by Frontier Laboratories Ltd.), detector (flame ionization detector (FID)), injection temperature: 300° C., detector temperature: 350° C., flow rate of He: 4.6 mL/min.

(ii) Method for Measuring the Ratio by Mass of an Alkenyl Sulfate to a Hydroxyalkyl Sulfate

The ratio by mass of an alkenyl sulfate to a hydroxyalkyl sulfate was measured by HPLC-MS. Specifically, the alkenyl sulfate was separated from the hydroxyalkyl sulfate and each was subjected to MS to identify. As a result, each ratio was found from the GC-MS peak area.

In this case, instruments and conditions used for the measurement are as follows.

HPLC apparatus (trade name: Agilent Technology 1100, manufactured by Agilent Technologies Inc.), column (trade name: L-column ODS 4.6×150 mm, manufactured by (General Incorporated Foundation) Chemical Evaluation and Research Institute, Japan), preparation of a sample: (diluted 1000-fold with methanol), eluent A (aqueous 10 mM ammonium acetate solution), eluent B (10 mM ammonium acetate addition methanol solution), gradient (0 min. (A/B=30/70%)→10 min. (30/70%)→55 min. (0/100%)→65 min. (0/100%)→66 min. (30/70%)→75 min. (30/70%)), MS instrument (trade name: Agilent Technology 1100MS SL (G1946D)), MS detection (detection of negative ions m/z 60-1600, UV240 nm).

(iii) Method for Measuring the Content of a Raw Material Internal Olefin

The content of the raw material internal olefin was measured by GC. Specifically, after ethanol and petroleum ether were added to an aqueous internal olefin sulfonate solution, the solution was subjected to extraction to obtain an olefin in the petroleum ether phase. As a result, the amount of an olefin was quantitatively measured from the GC peak area. The instruments and analysis conditions used in the measurement are as follows. GC instrument (trade name: Agilent Technology 6850, manufactured by Agilent Technologies Inc.), column (trade name: Ultra-Alloy-1HT Capillary Column 15 m×250 μm×0.15 μm, manufactured by Frontier Laboratories Ltd.), detector: (flame ionization detector (FID)), injection temperature: 300° C., detector temperature 350° C., flow rate of He 3.8 mL/min.

(iv) Method for Measuring the Content of an Inorganic Compound:

The content of an inorganic compound was measured by potentiometry or neutralization titration. Specifically, the content of Na₂SO₄ was quantitatively measured by finding the amount of a sulfate radical (SO₄⁻²) by potentiometry. Also, the content of NaOH was measured by neutralization titration using dilute hydrochloric acid.

(v) Method for Measuring the Content of a Paraffin Component

The content of a paraffin component was measured by GC. Specifically, after ethanol and petroleum ether were added to an aqueous internal olefin sulfonate solution, the solution was subjected to extraction to obtain a paraffin in the petroleum ether phase. As a result, the amount of a paraffin was quantitatively measured from the GC peak area. The instruments and analysis conditions used in the measurement are the same as those used for the measurement of the content of the raw material internal olefin.

### (2) Production of an Internal Olefin

### Production Example A

### Synthesis of an Internal Olefin in which the Number of Carbon Atoms is 16 and the Content of a Double Bond at C2-Position is 16.5% by Mass

A flask equipped with a stirrer was charged with 7000 g (28.9 mol) of 1-hexadecanol (trade name: Kalcol 6098, manufactured by Kao Corporation) and 700 g (10% by mass based on raw material alcohol) of γ-alumina (manufactured by STREM Chemicals, Inc.) as a solid acid catalyst and the mixture was reacted at 280° C. with stirring for 5 hours while flowing nitrogen (7000 mL/min.) in the system. The conversion rate of alcohol after the reaction was finished was 100% and the purity of a C₁₆ internal olefin was 99.7%.

The obtained crude alkene internal olefin was transferred to a distilling flask to distill at 136 to 160° C./4.0 mmHg, thereby obtaining an alkene internal olefin having 16 carbon atoms and an olefin purity of 100%.

The distribution of double bond in the obtained alkene internal olefin was as follows: C1-position: 0.5% by mass, C2-position: 16.5% by mass, C3-position: 15.4% by mass, C4-position: 16.4% by mass, C5-position: 17.2% by mass, C6-position: 14.2% by mass, and C7-position+C8-position: 19.8% by mass.

### Production Example B

### Synthesis of an Internal Olefin in which the Number of Carbon Atoms is 18 and the Content of a Double Bond at C2-Position was 16.9% by Mass

A flask equipped with a stirrer was charged with 7000 g (25.9 mol) of 1-octadecanol (trade name: Kalcol 8098, manufactured by Kao Corporation) and 1050 g (15% by mass based on raw material alcohol) of γ-alumina (manufactured by STREM Chemicals, Inc.) as a solid acid catalyst and the mixture was reacted at 285° C. with stirring for 13 hours while flowing nitrogen (7000 mL/min) in the system. The conversion rate of alcohol after the reaction was finished was 100% and the purity of a C₁₈ internal olefin was 98.5%.

The obtained crude internal olefin was transferred to a distilling flask to distill at 148 to 158° C./0.5 mmHg, thereby obtaining an internal olefin having 18 carbon atoms and an olefin purity of 100%.

The distribution of double bond in the obtained internal olefin was as follows: C1-position: 0.7% by mass, C2-position: 16.9% by mass, C3-position: 15.9% by mass, C4-position: 16.0% by mass, C5-position: 14.7% by mass, C6-position: 11.2% by mass, C7-position: 10.2% by mass, and C8-position+C9-position: 14.6% by mass.

### (3) Production of an Alkene Sulfonate and Hydroxyalkane Sulfonate

### Production Example 1

The C₁₆ internal olefin (content of an internal olefin with a double bond at C2-position is 16.5% by mass) which was obtained in Production Example A was charged in a thin-film type sulfonating reactor equipped with an outer jacket and sulfur trioxide gas was flowed in the reactor while flowing 20° C. cooling water through the reactor outer jacket to undergo a sulfonation reaction. The molar ratio of SO₃/internal olefin in the sulfonation reaction was set to 1.09.

The obtained sulfonated product was added in an aqueous alkali solution prepared by sodium hydroxide in an amount of 1.5 mol equivalent to theoretical acid value to neutralize at 30° C. for 1 hour with stirring.

The neutralized product was heated at 160° C. for 1 hour in an autoclave to hydrolyze, thereby obtaining a sodium C₁₆ olefin sulfonate crude product.

300 g of the crude product was transferred to a separatory funnel, to which 300 mL of ethanol was then added and then 300 mL of petroleum ether was added at a time to extract and remove oil-soluble impurities. At this time, inorganic compounds (major component: mirabilite) precipitated at the oil-water interface by the addition of ethanol was also removed from the water phase by a water-oil separation operation. This extraction and removing operation was performed three times. The water-phase side was evaporated to dryness to obtain sodium C₁₆ internal olefin sulfonate.

The ratio by mass of an alkene sulfonate to a hydroxyalkane sulfonate in the obtained sodium internal olefin sulfonate was 19/81. Also, the content of a raw material internal olefin contained in the obtained sodium internal olefin sulfonate was less than 100 ppm (less than the detection limit of GC) and the content of inorganic compounds was 1.3% by mass.

### Production Example 2

A sodium C₁₈ internal olefin sulfonate was obtained from the C₁₈ internal olefin (content of an internal olefin with a double bond at C2-position is 16.9% by mass) which was obtained in Production Example B, in the same condition as in Production Example 1.

The ratio by mass of an alkene sulfonate to a hydroxyalkane sulfonate in the obtained sodium C₁₈ internal olefin sulfonate was 20/80. Also, the content of a raw material internal olefin contained in the obtained sodium internal olefin sulfonate was less than 100 ppm (less than the detection limit of GC) and the content of inorganic compounds was 1.7% by mass.

### Examples 1 to 4 and Comparative Examples 1 to 4

Aqueous hair cleansing agents were prepared using the formulations shown in Table 1 and evaluated in the methods described below. The results of evaluation are shown together in Table 1. In this table, the pH shows a value at 25° C. when the hair cleansing agent was diluted 20-fold by mass with water.

### (Method of Evaluation of an Aqueous Hair Cleansing Agent)

Japanese women's hair which had been subjected to straight-permanent treatment once and bleaching treatment twice was regarded as damaged hair and four professional panelists treated 10 g of the damaged hair bound in a bun in the following method to make functional evaluation.

### (1) Foamability

The above damaged hair bound in a bun (length: 25 cm, width: 5.5 cm, and weight: 10 g) was lightly rinsed with 40° C. warm water, unnecessary water was removed, and 0.5 g of an aqueous hair cleansing agent was used to form ample foam for about 30 minutes. Then, the condition of foam was functionally evaluated according to the following five-rank rating. The evaluation was made by four panelists to calculate an average of the evaluations of four panelists.

5: very good foaming

4: good foaming

3: somewhat good foaming

2: not very good foaming

1: poor foaming

### (2) Smoothness in Rinsing

The above damaged hair bound in a bun (length: 25 cm, width: 5.5 cm, and weight: 10 g) was lightly rinsed with 40° C. warm water, unnecessary water was removed, and 0.5 g of an aqueous hair cleansing agent was used to form ample foam for about 30 minutes. After that, the hank of hair with foam was rinsed with 40° C. warm water flowing at a rate of 2 L/min to functionally evaluate the smoothness according to the following five-rank rating. The evaluation was made by four panelists to calculate an average of the evaluations of four panelists.

5: the surface of hair is very smooth.

4: the surface of hair is smooth.

3: the surface of hair is somewhat smooth.

2: the surface of hair is not very smooth.

1: the surface of hair is unsmooth.

(3) Shininess of Hair after Dried

The above damaged hair bound in a bun (length: 25 cm, width: 5.5 cm, and weight: 10 g) was lightly rinsed with 40° C. warm water, unnecessary water was removed, and 0.5 g of an aqueous hair cleansing agent was used to form ample foam for about 30 minutes. After that, the hank of hair with foam was rinsed with 40° C. warm water flowing at a rate of 2 L/min and then dried by a drier to functionally evaluate the shininess of the hair after the hair was completely dried according to the following five-rank rating. The evaluation was made by four panelists to calculate an average of the evaluations of four panelists.

5: very shiny

4: shiny

3: somewhat shiny

2: not very shiny

1: not shiny

TABLE 1

| | | Example | | | | Comparative Example | | | |
|---|---|---|---|---|---|---|---|---|---|
| (%) | | 1 | 2 | 3 | 4 | 1 | 2 | 3 | 4 |
| (A) | Sodium C₁₆ internal olefin sulfonate | 6.0 | — | 4.2 | 3.0 | — | 6.0 | 6.0 | 6.0 |
| | (Production Example 1) | | | | | | | | |
| | Sodium C₁₈ internal olefin sulfonate | — | 6.0 | 1.8 | — | — | — | — | — |
| | (Production Example 2) | | | | | | | | |
| (D) | Ammonium lauryl ether (1) sulfate*¹ | 6.0 | 6.0 | 6.0 | 9.0 | 12 | 6.0 | 6.0 | 6.0 |
| (B) | Benzyl alcohol | 0.3 | 0.3 | 0.3 | 0.3 | 0.3 | — | 0.3 | 0.3 |
| | Polypropylene (7) glycol: MW 420 | 1.0 | 1.0 | 1.0 | 1.0 | 1.0 | — | 1.0 | 1.0 |
| (C) | Malic acid | 0.55 | 0.55 | 0.55 | 0.55 | 0.55 | 0.55 | — | 0.55 |
| | Lactic acid | 0.20 | 0.20 | 0.20 | 0.20 | 0.20 | 0.20 | — | 0.20 |
| (E) | Isodecyl glyceryl ether*² | 0.4 | 0.4 | 0.4 | 0.4 | 0.4 | 0.4 | 0.4 | 0.4 |
| | Polyoxypropylene (3) monooctyl ether*³ | 0.7 | 0.7 | 0.7 | 0.7 | 0.7 | 0.7 | 0.7 | 0.7 |
| (F) | Laurylhydroxy sultaine*⁴ | 1.0 | 1.0 | 1.0 | 1.0 | 1.0 | 1.0 | 1.0 | 1.0 |
| Others | Polyoxyethylene(6) stearyl ether | 2.6 | 2.6 | 2.6 | 2.6 | 2.6 | 2.6 | 2.6 | 2.6 |
| | Lanoline fatty acid*⁵ | 0.01 | 0.01 | 0.01 | 0.01 | 0.01 | 0.01 | 0.01 | 0.01 |
| | Lauric acid | 0.4 | 0.4 | 0.4 | 0.4 | 0.4 | 0.4 | 0.4 | 0.4 |
| | Myristyl alcohol | 0.4 | 0.4 | 0.4 | 0.4 | 0.4 | 0.4 | 0.4 | 0.4 |
| | Ethylene glycol distearyl | 1.6 | 1.6 | 1.6 | 1.6 | 1.6 | 1.6 | 1.6 | 1.6 |
| | Guar hydroxypropyltrimonium chloride*⁶ | 0.4 | 0.4 | 0.4 | 0.4 | 0.4 | 0.4 | 0.4 | 0.4 |
| | Polyquaternium-10*⁷ | 0.1 | 0.1 | 0.1 | 0.1 | 0.1 | 0.1 | 0.1 | 0.1 |
| | Polyquaternium-7*⁸ | 0.2 | 0.2 | 0.2 | 0.2 | 0.2 | 0.2 | 0.2 | 0.2 |
| | Dimethylpolysiloxane emulsion*⁹ | 1.0 | 1.0 | 1.0 | 1.0 | 1.0 | 1.0 | 1.0 | 1.0 |
| | Sodium chloride | 0.4 | 0.4 | 0.4 | 0.4 | 0.4 | 0.4 | 0.4 | 0.4 |
| | Sodium hydroxide | (*10) | (*10) | (*10) | (*10) | (*10) | (*10) | (*10) | (*10) |
| | Purified water | Balance | Balance | Balance | Balance | Balance | Balance | Balance | Balance |
| pH | | 3.7 | 3.7 | 3.7 | 3.7 | 3.7 | 3.7 | 3.7 | 6 |
| Rating | Foamability | 4 | 3 | 3 | 4.5 | 5 | 4 | 4 | 4 |
| | Smoothness in rinsing | 4.5 | 5 | 5 | 3 | 2 | 2 | 2 | 2 |
| | shininess of hair after dried | 5 | 5 | 4.5 | 3 | 2 | 2 | 2 | 2 |

*¹EMAL 125A (manufactured by Kao Corporation)
*²PENETOL GE-ID (manufactured by Kao Corporation)
*³KAO SOFCARE GP (manufactured by Kao Corporation)
*⁴ANHITOL 20HD (manufactured by Kao Corporation)
*⁵Lanolin Fatty Acid (manufactured by Croda Europe Ltd.)
*⁶Jaguar C-14S (manufactured by Rhodia)
*⁷POIZ M-80 (manufactured by Kao Corporation)
*⁸MARCOAT550 (manufactured by Lubrizol Advanced Materials, Inc.)
*⁹Silicone CF2460 (manufactured by Dow Corning Toray Co., Ltd.)
(*10) Amount for pH regulation

As is clear from Table 1, Comparative Example 1 including no component (A), Comparative Example 2 including no component (B), Comparative Example 3 including no component (C), and Comparative Example 4 having a pH of 6 were deteriorated particularly in smoothness in rinsing and in shininess after the hair was dried. Examples 1 to 4 of the present invention each had good results. Among these examples, Examples 2 and 3 each containing the component (A) having 18 carbon atoms was slightly more deteriorated in foaming as compared with Example 1 containing the component (A) having 16 carbon atoms and Example 4 reduced in the component (A) was slightly deteriorated in smoothness in rinsing and in shininess.

### Examples 5 to 12

Aqueous hair cleansing agents using the formulations shown in Table 2 were prepared to evaluate “(4) quickness of foaming” described below besides the evaluation of the aforementioned (1) to (3). Example 1 shown in Table 1 was also evaluated according to the above (4). The results of evaluation are shown together in Table 2. The pH shows a value at 25° C. when the hair cleansing agent was diluted 20-fold by mass with water.

### (4) Quickness of Foaming

The above damaged hair bound in a bun (length: 25 cm, width: 5.5 cm, and weight: 10 g) was lightly rinsed with 40° C. warm water, unnecessary water was removed, and 0.5 g of an aqueous hair cleansing agent was used to form ample foam for about 30 seconds to functionally evaluate the quickness of foaming according to the following five-rank rating. The evaluation was made by four panelists to calculate an average of the evaluations of four panelists.

5: the detergent forms foam very quickly.

4: the detergent forms foam quickly.

3: the detergent forms foam somewhat quickly.

2: the detergent does not form foam so quickly.

1: the detergent does not form foam quickly.

TABLE 2

| | | Example | | | | | | | | |
|---|---|---|---|---|---|---|---|---|---|---|
| (%) | | 1 | 5 | 6 | 7 | 8 | 9 | 10 | 11 | 12 |
| (A) | Sodium C₁₆ internal olefin sulfonate | 6.0 | 6.0 | 6.0 | 6.0 | 6.0 | 6.0 | 6.0 | 6.0 | 6.0 |
| | (production Example 1) | | | | | | | | | |
| (D) | Ammonium lauryl ether (1) sulfate*¹ | 6.0 | — | 6.0 | 6.0 | 6.0 | 6.0 | 6.0 | 6.0 | — |
| | Ammonium lauryl ether (2) sulfate*¹¹ | — | 6.0 | — | — | — | — | — | — | 6.0 |
| (B) | Benzyl alcohol | 0.3 | 0.3 | 0.1 | 0.3 | 0.3 | 0.3 | 0.3 | 0.3 | 0.3 |
| | Polypropylene (7) glycol: MW 420 | 1.0 | 1.0 | 0.1 | 1.0 | 1.0 | 1.0 | 1.0 | 1.0 | 1.0 |
| (C) | Malic acid | 0.55 | 0.55 | 0.55 | 0.10 | 0.55 | 0.55 | 0.55 | 0.55 | 0.55 |
| | Lactic acid | 0.20 | 0.20 | 0.20 | 0.04 | 0.20 | 0.20 | 0.20 | 0.20 | 0.20 |
| (E) | Isodecyl glyceryl ether*² | 0.4 | 0.4 | 0.4 | 0.4 | 0.04 | 1.60 | 0.4 | 0.4 | 0.4 |
| | Polyoxypropylene (3) monooctyl ether*³ | 0.7 | 0.7 | 0.7 | 0.7 | 0.07 | — | 0.7 | 0.7 | 0.7 |
| (F) | Laurylhydroxy sultaine*⁴ | 1.0 | 1.0 | 1.0 | 1.0 | 1.0 | 1.0 | — | 0.1 | — |
| | Lauramidopropyl betaine*¹² | — | — | — | — | — | — | 1.0 | — | 1.0 |
| Others | Polyoxyethylene (6) stearyl ether | 2.6 | 2.6 | 2.6 | 2.6 | 2.6 | 2.6 | 2.6 | 2.6 | 2.6 |
| | Lanoline fatty acid*⁵ | 0.01 | 0.01 | 0.01 | 0.01 | 0.01 | 0.01 | 0.01 | 0.01 | 0.01 |
| | Lauric acid | 0.4 | 0.4 | 0.4 | 0.4 | 0.4 | 0.4 | 0.4 | 0.4 | 0.4 |
| | Myristyl alcohol | 0.4 | 0.4 | 0.4 | 0.4 | 0.4 | 0.4 | 0.4 | 0.4 | 0.4 |
| | Ethylene glycol distearyl | 1.6 | 1.6 | 1.6 | 1.6 | 1.6 | 1.6 | 1.6 | 1.6 | 1.6 |
| | Guarhydroxypropyltrimoniumchloride*⁶ | 0.4 | 0.4 | 0.4 | 0.4 | 0.4 | 0.4 | 0.4 | 0.4 | 0.4 |
| | Polyquaternium-10*⁷ | 0.1 | 0.1 | 0.1 | 0.1 | 0.1 | 0.1 | 0.1 | 0.1 | 0.1 |
| | Polyquaternium-7*⁸ | 0.2 | 0.2 | 0.2 | 0.2 | 0.2 | 0.2 | 0.2 | 0.2 | 0.2 |
| | Dimethylpolysiloxane emulsion*⁹ | 1.0 | 1.0 | 1.0 | 1.0 | 1.0 | 1.0 | 1.0 | 1.0 | 1.0 |
| | Sodium chloride | 0.4 | 0.4 | 0.4 | 0.4 | 0.4 | 0.4 | 0.4 | 0.4 | 0.4 |
| | Sodium hydroxide | (*10) | (*10) | (*10) | (*10) | (*10) | (*10) | (*10) | (*10) | (*10) |
| | Purified water | Balance | Balance | Balance | Balance | Balance | Balance | Balance | Balance | Balance |
| pH | | 3.7 | 3.7 | 3.7 | 3.7 | 3.7 | 3.7 | 3.7 | 3.7 | 3.7 |
| Rating | Foamability | 4 | 4.5 | 4 | 4 | 4 | 4 | 4.5 | 2.5 | 5 |
| | Quickness of foaming | 4 | 4.5 | 4 | 4 | 2.5 | 4.5 | 4.5 | 2.5 | 5 |
| | Smoothness in rinsing | 4.5 | 5 | 4.5 | 4.5 | 4.5 | 4.5 | 4.75 | 4.5 | 5 |
| | shininess of hair after dried | 5 | 5 | 3 | 2.5 | 5 | 5 | 5 | 5 | 5 |

*¹¹EMAL 270S (manufactured by Kao Corporation)
*¹²ANHITOL 20AB (manufactured by Kao Corporation)

### Examples 13 to 17

An aqueous hair cleansing agent according to the present invention was prepared by using the following formulations. The pH shows a value at 25° C. when the hair cleansing agent was diluted 20-fold by mass with water.

### Example 13

### Cleansing Shampoo (pH 4.2)

| | (%) |
|---|---|
| Sodium C₁₆ internal olefin sulfonate | 8 |
| (Production Example 1) | |
| Sodium C₁₈ internal olefin sulfonate | 3 |
| (Production Example 2) | |
| Polyoxyethylene (16) lauryl ether | 2 |
| Polyoxyethylene (4) lauryl ether | 0.5 |
| Benzyl alcohol | 0.5 |
| Lactic acid | 1 |
| Citric acid | 0.1 |
| Isodecyl glyceryl ether*² | 1 |
| Polyquaternium-10*⁷ | 0.1 |
| Sodium benzoate | 0.1 |
| Sodium chloride | 0.4 |
| 48% Sodium hydroxide | Adjusted to pH 4.2 |
| Perfume | Proper amount |
| Purified water | Balance |

The shampoo of Example 13 is superior in the foaming of a detergent, has good characteristics ability, and is able to provide shininess to hair.

### Example 14

### Conditioning Shampoo (Non-Silicon Type) (pH 3.9)

| | (%) |
|---|---|
| Sodium C₁₆ internal olefin sulfonate | 8 |
| (Production Example 1) | |
| Sodium C₁₈ internal olefin sulfonate | 3 |
| (Production Example 2) | |
| Polyoxyethylene (16) lauryl ether | 2 |
| Polyoxyethylene (4) lauryl ether | 0.5 |
| Laurylhydroxy sultaine*⁴ | 0.5 |
| Lauryl monoethanolamide | 0.2 |
| Benzyl alcohol | 0.5 |
| Malic acid | 0.5 |
| Lactic acid | 0.5 |
| Glycolic acid | 0.1 |
| Ethylene glycol distearate | 1.0 |
| Isodecyl glyceryl ether*² | 1 |
| Polyoxypropylene (3) monooctyl ether*³ | 0.5 |
| Guar hydroxypropyltrimonium chloride*⁶ | 0.3 |
| Polyquaternium-10*⁷ | 0.1 |
| Polyquaternium-52 | 0.05 |
| Lanolin fatty acid*⁵ | 0.02 |
| Sodium chloride | 0.3 |
| 48% Sodium hydroxide | Adjusted to pH 3.9 |
| Perfume | Proper amount |
| Purified water | Balance |

This shampoo is superior in the foaming of a detergent, has good rinsing characteristics, and is able to provide shinineess to hair.

### Example 15

### Conditioning Shampoo (pH 5.0)

| | (%) |
|---|---|
| Sodium C₁₆ internal olefin sulfonate | 6 |
| (Production Example 1) | |
| Ammonium lauryl ether (1) sulfate | 6 |
| Polyoxyethylene (4) lauryl ether | 0.5 |
| Lauramidopropyl betaine*¹² | 2 |
| Laurylmonoethanolamide | 0.2 |
| Benzyl alcohol | 0.5 |
| Polypropylene (7) glycol (molecular weight: | 0.5 |
| 420) (trade name: ADEKA KARPOL DL-30, | |
| manufactured by ADEKA CORPORATION) | |
| Malic acid | 0.5 |
| Lactic acid | 0.5 |
| Glycolic acid | 0.1 |
| Isodecyl glyceryl ether*² | 1 |
| Ethylene glycol distearate | 1 |
| Myristyl alcohol | 0.5 |
| Guar hydroxypropyltrimonium chloride*⁶ | 0.3 |
| Polyquaternium-10*⁷ | 0.1 |
| Polyquaternium-7*⁸ | 0.1 |
| Lanolin fatty acid*⁵ | 0.02 |
| Amodimethicone | 0.2 |
| Sodium chloride | 0.3 |
| Sodium benzoate | 0.1 |
| 48% Sodium hydroxide | Adjusted to pH 5.0 |
| Perfume | Proper amount |
| Purified water | Balance |

This shampoo is superior in the foaming of a detergent, has good rinsing characteristics, and is able to give luster to hair.

### Example 16

### Anti-Dandruff Shampoo (pH 5.0)

| | (%) |
|---|---|
| Sodium C₁₆ internal olefin sulfonate | 11 |
| (Production Example 1) | |
| Polyoxyethylene (4) lauryl ether | 0.5 |
| Laurylhydroxy sultaine*⁴ | 2 |
| Laurylmonoethanolamide | 0.2 |
| Polypropylene (7) glycol (molecular weight: | 0.5 |
| 420) (trade name: ADEKA KARPOL DL-30, | |
| manufactured by ADEKA CORPORATION) | |
| Malic acid | 0.5 |
| Citric acid | 0.1 |
| Isodecyl glyceryl ether*² | 0.5 |
| Polyoxypropylene (3) monooctyl ether*³ | 0.5 |
| Myristyl alcohol | 0.5 |
| Guar hydroxypropyltrimonium chloride*⁶ | 0.2 |
| Zinc pyrithione | 1 |
| Dimethicone (trade name: CF2460, | 1 |
| manufactured by Dow Corning | |
| Toray Co., Ltd.) | |
| Sodium chloride | 0.3 |
| Sodium benzoate | 0.1 |
| 48% Sodium hydroxide | Adjusted to pH 5.0 |
| Perfume | Proper amount |
| Purified water | Balance |

This shampoo is superior in the foaming of a detergent, has good rinsing characteristics, and is able to provide shininess to hair.

### Example 17

### Anti-Dandruff Shampoo (pH 3.7)

| | (%) |
|---|---|
| Sodium C₁₆ internal olefin sulfonate | 1 |
| (Production Example 1) | |
| Ammonium lauryl ether (1) sulfate | 10 |
| Polyoxyethylene (16) lauryl ether | 1 |
| Polyoxyethylene (4) lauryl ether | 0.2 |
| Laurylhydroxy sultaine*⁴ | 2 |
| Laurylmonoethanolamide | 0.2 |
| Polypropylene (7) glycol (molecular weight: | 0.5 |
| 420) (trade name: ADEKA KARPOL DL-30, | |
| manufactured by ADEKA CORPORATION) | |
| Isodecyl glyceryl ether*² | 0.5 |
| Polyoxypropylene (3) monooctyl ether*³ | 0.5 |
| Ethylene glycol distearate | 1 |
| Myristyl alcohol | 0.5 |
| Polyquaternium-10*⁷ | 0.3 |
| Dipotassium glycyrrhizinate | 0.1 |
| Sodium chloride | 0.3 |
| Malic acid | 1 |
| Citric acid | 0.1 |
| Perfume | Proper amount |
| 48% sodium hydroxide | Adjusted to pH 3.7 |
| Purified water | Balance |

This shampoo is superior in the foaming of a detergent, has good rinsing characteristics, and is able to provide shininess to hair.

1. An aqueous hair cleansing agent comprising the following components (A), (B), (C), and water and having a pH of 2 to 5 at 25° C. when the hair cleansing agent was diluted 20-fold by mass with water:
(A) an internal olefin sulfonate;
(B) at least one organic solvents selected from the group consisting of the following (B1) to (B5);
(B1) an aromatic alcohol represented by the following formula (1): 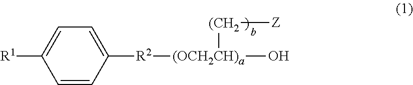
wherein R¹ represents a hydrogen atom, a methyl group, or a methoxy group, R² represents a single bond or a straight-chain or a branched chain alkylene group or alkenylene group having 1 to 3 carbon atoms, Z represents a hydrogen atom or a hydroxyl group, and a and b respectively denote a number from 0 to 5;
(B2) a polypropylene glycol having a molecular weight of 200 to 1000;
(B3) a N-alkyl pyrrolidone in which an alkyl group having 1 to 18 carbon atoms is bound with a nitrogen atom;
(B4) an alkylene carbonate having 1 to 4 carbon atoms; and
(B5) a lactone or a cyclic ketone represented by the following formula (2), (3), or (4): 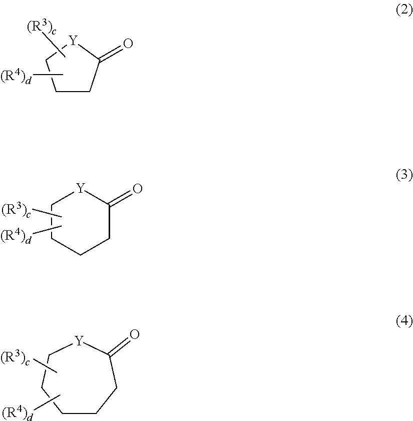
wherein Y represents a methylene group or an oxygen atom, R³ and R⁴ represent substituents differing from each other, and c and d denote a number from 0 or 1; and
(C) at least one organic carboxylic acids selected from the group consisting of a hydroxymonocarboxylic acid and dicarboxylic acid.

2. The aqueous hair cleansing agent according to claim 1, wherein the internal olefin sulfonate comprises:
(A1) an alkene sulfonic acid containing a sulfonyl group on any one of the carbon atoms of a straight-chain alkene having 12 or more and 24 or less carbon atoms excluding terminal carbon atoms or its salt;
(A2) a hydroxy alkane sulfonic acid containing a sulfonyl group on anyone of the carbon atoms of a straight-chain hydroxy alkane having 12 or more and 24 or less carbon atoms excluding terminal carbon atoms or its salt; or a mixture of these compounds.

3. The aqueous hair cleansing agent according to claim 2, wherein a ratio (A2)/(A1) by mass of the component (A2) to the component (A1) in the component (A) is preferably 50/50 to 95/5 and more preferably 50/50 to 90/10.

4. The aqueous hair cleansing agent according to claim 1, wherein the component (A) preferably comprises an internal olefin sulfonate having 16 carbon atoms and an internal olefin sulfonate having 18 carbon atoms.

5. The aqueous hair cleansing agent according to claim 1, wherein the ratio (internal olefin sulfonate having 16 carbon atoms/internal olefin sulfonate having 18 carbon atoms) by mass of the content of an internal olefin sulfonate having 16 carbon atoms to the content of an internal olefin sulfonate having 18 carbon atoms is preferably 50/50 to 99/1, more preferably 60/40 to 95/5, even more preferably 70/30 to 90/10, even more preferably 75/25 to 90/10, and even more preferably 75/25 to 85/15.

6. The aqueous hair cleansing agent according to claim 1, wherein the total ratio of the internal olefin sulfonates having 16 and 18 carbon atoms in the total amount of the component (A) is preferably 50% by mass or more, more preferably 60% by mass, even more preferably 70% by mass, even more preferably 80% by mass or more, even more preferably 90% by mass or more, and even more preferably 95% by mass or more.

7. The aqueous hair cleansing agent according to claim 1, wherein the content of a material with a sulfonic acid group located at the second position thereof in the component (A) is preferably 20% by mass or less, more preferably less than 20% by mass, even more preferably 19% by mass or less, even more preferably 18% by mass or less, and even more preferably 17.5% by mass or less, and 5% by mass or more, preferably 6% by mass or more, more preferably 7% by mass or more, even more preferably 8% by mass or more, and even more preferably 9% by mass or more.

8. The aqueous hair cleansing agent according to claim 1, wherein the content of an internal olefin sulfonate in which a sulfonic acid group is located at the third position or more inward position of the olefin chain or alkane chain in the component (A) is preferably 70% by mass or more, more preferably 75% by mass or more, and even more preferably 80% by mass or more.

9. The aqueous hair cleansing agent according to claim 1, wherein the content of the component (A) is preferably 1 to 20% by mass, more preferably 2 to 18% by mass, and even more preferably 5 to 15% by mass or less relative to the total amount of the aqueous hair cleansing agent.

10. The aqueous hair cleansing agent according to claim 1, wherein the component (B) is at least one selected from the group consisting of benzyl alcohol, 2-benzyloxyethanol, and a polypropylene glycol having a molecular weight of 200 to 700.

11. The aqueous hair cleansing agent according to claim 1, wherein the content of the component (B) is preferably 0.01% by mass or more, more preferably 0.05% by mass or more, and even more preferably 0.1% by mass or more, and 20% by mass or less, preferably 10% by mass or less, and more preferably 5% by mass or less relative to the total amount of the aqueous hair cleansing agent.

12. The aqueous hair cleansing agent according to claim 1, wherein the content of the component (C) is preferably 0.3% by mass or more, more preferably 0.5% by mass or more, and even more preferably 0.7% by mass or more, and 5% by mass or less, preferably 3% by mass or less, and more preferably 2% by mass or less relative to the total amount of the aqueous hair cleansing agent.

13. The aqueous hair cleansing agent according to claim 1, wherein the component (C) is one or more types selected from the group consisting of lactic acid, glycolic acid, malic acid, and tartaric acid.

14. The aqueous hair cleansing agent according to claim 1, wherein the content of water is preferably 50% by mass or more and 95% by mass or less and more preferably 60% by mass or more and 90% by mass or less relative to the total amount of the aqueous hair cleansing agent.

15. The aqueous hair cleansing agent according to claim 1, the hair cleansing agent further comprising, as a component (D), a polyoxyethylene alkyl ether sulfate represented by the following formula (5):
R⁵O(CH₂CH₂O)ₑSO₃M (5)
wherein R⁵ represents an alkyl group or an alkenyl group having 10 to 18 carbon atoms, M represents an alkali metal, an alkali earth metal, ammonium, an alkanol amine, or a basic amino acid, and e represents an average addition molar number of ethylene oxide and denotes a number from 0.5 to 5 in an average by mass.

16. The aqueous hair cleansing agent according to claim 15, wherein the total amount of the components (A) and (D) is preferably 3% by mass or more, more preferably 5% by mass or more, and even more preferably 7% by mass or more, and 20% by mass or less, preferably 19% by mass or less, and even more preferably 18% by mass or less relative to the total amount of the aqueous hair cleansing agent.

17. The aqueous hair cleansing agent according to claim 1, the hair cleansing agent further comprising, as a component (E), a nonionic surfactant selected from a glyceryl ether represented by the following formula (6) and a polyoxyalkylene alkyl ether represented by the formula (7):
R⁶—O—CH₂—CH(OH)—CH₂—OH (6)
wherein R⁶ represents a straight-chain or branched hydrocarbon group having 1 to 22 carbon atoms;
R⁷O—(PO)ₙ/(EO)ₘ—R⁸ (7)
wherein R⁷ represents a straight-chain alkyl group or alkenyl group having 8 to 10 carbon atoms, PO represents a propyleneoxy group, EO represents an ethyleneoxy group, n which is an average addition molar number denotes a number from 1.5 to 3.0, m which is an average addition molar number denotes a number from 0 to 1.0, and R⁸ represents a hydrogen atom or a methyl group.

18. The aqueous hair cleansing agent according to claim 17, wherein the content of the component (E) is preferably 0.01 to 20% by mass, more preferably 0.05 to 15% by mass, and more preferably 0.1 to 10% by mass relative to the total amount of the aqueous hair cleansing agent.

19. The aqueous hair cleansing agent according to claim 1, the hair cleansing agent further comprising, as a component (F), an amphoteric surfactant.

20. The aqueous hair cleansing agent according to claim 19, wherein the content of the component (F) is preferably 0.01 to 15% by mass, more preferably 0.05 to 8% by mass, and more preferably 0.1 to 6% by mass relative to the total amount of the aqueous hair cleansing agent.
